(19) United States
(12) Patent Application Publication
(10) Pub. No.: US 2024/0024171 A1
(43) Pub. Date: Jan. 25, 2024

(54) METHOD FOR PROVIDING APERTURES IN A NONWOVEN WEB, AND APERTURED NONWOVEN WEB

(71) Applicant: The Procter & Gamble Company, Cincinnati, OH (US)

(72) Inventors: Rong DENG, Mason, OH (US); Craig POWELL, Independence, KY (US)

(21) Appl. No.: 18/351,694

(22) Filed: Jul. 13, 2023

Related U.S. Application Data
(60) Provisional application No. 63/390,312, filed on Jul. 19, 2022.

(51) Int. Cl. A61F 13/512; A61F 13/15

(52) U.S. Cl. CPC A61F 13/5126; A61F 13/15731; A61F 2013/51028

(57) ABSTRACT

A single step method for imparting a pattern of apertures in a fibrous nonwoven web is disclosed. The method may include providing a pinned roller bearing a pattern of aperturing pins thereon; providing an opposing roller; arranging the rollers so as to form a nip therebetween, wherein the nip has effectively zero clearance between top surfaces of the pins and outermost surfaces of the opposing roller; providing heating energy to one or both of rollers; rotating the rollers and conveying the nonwoven web through the nip, whereby material constituting fibers of the nonwoven web is at least partially melted and expressed from regions between the top surfaces of the pins and the opposing roller, and accumulates on the web about perimeter edges of the pin top surfaces. Resulting features of the nonwoven web, and products in which it may be incorporated as a component, are also disclosed.

### BACKGROUND

Disposable absorbent articles such as disposable baby diapers, absorbent training pants, incontinence pads, absorbent incontinence underwear, feminine hygiene pads/sanitary napkins, etc. typically include an arrangement of a liquid-permeable, wearer-facing topsheet, a liquid impermeable, outward-facing backsheet or barrier layer, and an absorbent structure disposed between the topsheet and the backsheet. The topsheet and the backsheet are typically bonded to each other about and to the outside of the perimeter of the absorbent structure, thereby enveloping and containing the components of the absorbent structure.

Web materials to be used to form topsheets have included polymer films and nonwoven web materials.

When a film is used to form a topsheet, typically, it will be formed or be imparted with a pattern of apertures therethrough, that provide passageways through which fluid can pass, from a wearer-facing surface to an opposing surface and down to components of the absorbent structure beneath.

Being formed from a batt of somewhat randomly-oriented filaments and/or fibers consolidated to form a fabric-like web, the typical nonwoven web material is, in many examples, inherently liquid permeable because the filaments and/or fibers (collectively, “fibers”), even when consolidated, do not form a continuous film-like barrier, but rather, a porous network or matrix of the randomly-oriented fibers, with interstitial spaces between them that provide passageways within the nonwoven structure through which fluid may pass.

Depending, however, upon the filament and/or fiber size, filament and/or fiber composition, nonwoven basis weight, and density of accumulation and consolidation of filaments and within the nonwoven structure, a nonwoven web material may in some examples be somewhat resistant to passage of fluid therethrough. For example, if a nonwoven is formed of constituent fibers that are relatively fine (i.e., of relatively low decitex or denier and/or relatively low average diameter/size), are relatively densely consolidated, and have relatively hydrophobic surface chemistry, the nonwoven may tend to resist passage of aqueous liquid therethrough, under circumstances of intended use.

It will be appreciated that in some circumstances, the designer of an absorbent article may wish to incorporate a wearer-facing topsheet formed of a nonwoven having a fiber constituency that provides a desired level of, e.g., softness attributes and/or pliability, opacity, and resistance to rewetting by fluid that has been transferred to absorbent structure components below. These attributes can be imparted to the topsheet nonwoven material by inclusion of, for example, relatively fine constituent fibers having hydrophobic surface chemistries, that are relatively densely consolidated in the nonwoven. Such a nonwoven material might resist passage of fluid therethrough, or may allow passage of fluid therethrough only at an unacceptably slow rate, under circumstances of intended use.

To increase the liquid permeability of such a topsheet nonwoven material, the product designer may choose to impart the nonwoven material with a pattern of apertures. The apertures are holes through the nonwoven material that are relatively larger than the inter-fiber spaces (often, by at least an order of magnitude), and are typically readily visible and distinguishable to the naked eye, viewing the surface of the nonwoven material. The apertures provide relatively larger passageways through which fluid may more easily pass. Several process technologies have developed for imparting nonwoven web materials with such apertures.

In one approach, apertures are simply cold-punched through the nonwoven web material. This approach has the drawback of reducing the structural integrity (tensile strength) of the web material resulting from cutting of fibers at the aperture edges, and making the web more prone to fuzzing or pilling, fiber shedding and fraying.

In another approach, a spunbond web of polymer fiber constituents may be passed through the nip between a pair of calender rollers, one or both of which may be heated and configured to impress a pattern of small, discrete compressed and fused regions, elongate along the machine direction, onto/into the web. The regions are of reduced caliper and consist of compressed and fused polymer material, from which the constituent fibers had been spun. Following such step, the web is passed through the nip between a pair of ring rollers (also known as grooved rollers) to incrementally stretch the web in the cross direction, which causes the fused regions to fracture and open along the cross direction, creating holes or apertures through the web. Following ring rolling the web is passed over a stretching roller and further stretched in the cross direction, to remove (pull out) rugosities that have been imparted by the ring rollers. Examples of this process are described in U.S. Pat. Nos. 5,916,661 and 10,667,962. Drawbacks of this process may include substantial reduction of structural integrity and tensile strength of the web material (particularly in the cross direction) and reduction of web opacity. Additionally, the process is typically unsuitable for nonwoven webs formed primarily or substantially of staple fibers, because the ring rolling and further stretching operations pull the constituent fibers apart, substantially degrading if not effectively destroying the web.

In another approach, an apertured web may be created by passing the precursor nonwoven web having polymer fiber constituents, through the nip between a pair of pin-and-socket rollers. The rollers include an aperturing roller bearing a pattern of radially outwardly-projecting, tapered aperturing pins, and an opposing, mating receiving roller bearing a corresponding pattern of pin receiving sockets, configured to receive the pins and also permit the rollers to rotate together without interference between the pins and the walls of the receiving sockets, as the pins are rotated through the nip. The aperturing roller may be heated. When the nonwoven web passes through the nip, the aperturing pins penetrate through the web and displace and separate the fibers of the web about them, along x-y directions relative the web. The heat from the aperturing roller softens polymeric components of the fibers and thereby helps effect permanent plastic deformation thereof, so that they remain in their displaced positions and define apertures, following exit of the web from the nip. Disadvantages associated with this process include limits on processing/throughput speed.

In still another approach, apertures may be created by hydrojetting or needling with hydrojets. This process does not cut, heat, substantially plastically deform or melt fibers, but rather, causes them to be displaced within the nonwoven, to open and form the holes. This approach also has several drawbacks. The fibers, being displaced but not plastically deformed to their displaced positions, may tend to return to the pre-displacement positions and/or otherwise shift into the spaces occupied by the holes, upon further downstream handling of the web material—the apertures and aperture pattern are not stable. Additionally, a hydrojetting process requires substantial energy input, in providing the water and pressure to the jets sufficient to achieve the desired effect, in required removal/drying of the water from the web following hydrojetting, and in collecting and further processing the removed water. Drawbacks associated with this process also include limits on processing/throughput speed.

Accordingly, opportunities for development of efficient and effective processes for aperturing nonwoven web materials, with efficient processing/throughput speed and preservation of structural integrity, desired aperture shape and patterning, remain.

### BRIEF DESCRIPTION OF THE FIGURES

FIG. 1 is a schematic plan view depiction of an example of an absorbent article in the form of a feminine hygiene pad.

FIG. 2 is a schematic lateral cross section of the feminine hygiene pad of FIG. 8, taken through line 2-2 in FIG. 1.

FIG. 3 is a schematic perspective depiction of a pair of aperturing rollers in operation upon a nonwoven web.

FIG. 4 is a schematic side view depiction of equipment on a manufacturing line including a pair of aperturing rollers in operation upon a nonwoven web.

FIGS. 5A and 5B are schematic side view depictions of differing alternative non-limiting examples of pairs of aperturing rollers.

FIGS. 6A and 6B are respective schematic mid-section, and perspective, views of an example of an aperturing pin.

FIG. 7A is a schematic magnified cross-section (taken along a y-z plane) depiction of an aperturing pin operating on a portion of a nonwoven web in a nip between aperturing rollers.

FIG. 7B is a schematic magnified cross-section (taken along a y-z plane) depiction of an aperturing pin operating on a portion of a nonwoven web in a nip between aperturing rollers, in another example.

FIG. 7C is a schematic magnified cross-section (taken along a y-z plane) depiction of an aperturing pin operating on a portion of a nonwoven web in a nip between aperturing rollers, in another example.

FIG. 8A is a schematic magnified cross-section (taken along a y-z plane) depiction of a portion of a nonwoven web having an aperture therethrough.

FIG. 8B is a schematic magnified cross-section (taken along a y-z plane) depiction of a portion of a nonwoven web having an aperture therethrough, in another example.

FIG. 9 is a schematic further magnified cross-section (taken along a y-z plane) depiction of the portion of the nonwoven web shown within circle 8 shown in FIG. 7.

FIG. 10A is a schematic magnified plan view (along a z-direction) depiction of the portion of the nonwoven web including an aperture.

FIG. 10B is a magnified photograph of a portion of a nonwoven web following its exit from a nip between an aperturing roller and an opposing roller.

FIG. 10C is a further magnified photograph of a portion of a nonwoven web following its exit from a nip between an aperturing roller and an opposing roller.

FIG. 11 is a plan view (along a z-direction) image of a portion of a nonwoven web material having a pattern of apertures therethrough.

FIG. 12 is a plan view (along a z-direction) magnified image of a portion of a nonwoven web material having an aperture therethrough.

FIG. 13 is a plan view (along a z-direction) magnified image of a portion of a nonwoven web material having an aperture therethrough.

### DESCRIPTION OF EXAMPLES

### Definitions

As used herein, the following terms shall have the meaning specified thereafter:

“Absorbent article” refers to disposable wearable devices, which absorb and/or contain liquid, and more specifically, refers to devices, which are placed against or in proximity to the body of the wearer to absorb and contain the various exudates discharged from the body. Absorbent articles can include diapers, training pants, adult incontinence undergarments (e.g., liners, pads and briefs) and/or feminine hygiene products, including feminine hygiene pads (also known as, for example, “sanitary napkins”, “menstrual pads”, “panty liners”, etc.). Such disposable devices are typically not manufactured of materials adapted to withstand laundering, such as knitted or woven fabrics.

The term “integrated” as used herein is used to describe fibers of a nonwoven material which have been intertwined, entangled, and/or pushed/pulled in a positive and/or negative Z-direction (direction of the thickness of the nonwoven material). Some exemplary processes for integrating fibers of a nonwoven web include spunlacing and needlepunching. Spunlacing (also known as “hydroentangling” or (“hydroenhancing”) uses a plurality of high pressure water jets directed at a precursor batt or accumulation of fibers being conveyed along a machine direction, to entangle the fibers. Needlepunching (also known as “needling”) involves the use of specially-featured needles to mechanically push and/or pull fibers, of a precursor batt or accumulation of fibers, in a z-direction, to entangle them with other fibers in the batt or accumulation.

The term “carded” as used herein is used to describe structural features of particular types of nonwoven web materials contemplated herein for use in some examples as apertured topsheet material. A carded nonwoven web is formed of fibers which are cut to a specific finite length, otherwise known as “staple length fibers.” Staple length fibers may be of any selected length. For example, staple length fibers may be cut to a length of up to 120 mm, to a length as short as 10 mm. However, if fibers of a particular group are staple length fibers, then the length of each of the fibers in the carded nonwoven is approximately the same, i.e. the staple length. Where fibers of more than one composition are included in a nonwoven web, for example, a web including polypropylene fibers and viscose fibers, the length of each fiber of the same composition may be substantially the same, while the respective staple fiber lengths of the respective fiber compositions may differ.

In contrast to staple fibers, filaments such as those produced by spinning, e.g., in a spunbond or meltblown nonwoven web manufacturing processes, are not ordinarily staple length fibers. Instead, these filaments are sometimes characterized as “continuous” fibers, meaning that they are of a relatively long and indeterminate length, not cut to a specific length following spinning, as their staple fiber counterparts are.

“Lateral”—with respect to an absorbent article such as a feminine hygiene pad, or a component thereof, refers to a direction parallel to a horizontal line tangent to the front surfaces of the upper portions of wearer's legs proximate the torso, when the pad is being worn normally and the wearer has assumed an even, square, normal standing position. A “width” dimension of any component or feature of an article such as a feminine hygiene pad is measured along the lateral direction. When the article or component thereof is laid out flat on a horizontal surface, the “lateral” direction corresponds with the lateral direction relative the structure when it is worn, as defined above. With respect to an article such as a feminine hygiene pad that is opened and laid out flat on a horizontal planar surface, “lateral” refers to a direction perpendicular to the longitudinal direction and parallel to the horizontal planar surface. With respect to an absorbent article, the “x-direction” is also the lateral direction.

The “lateral axis” of an absorbent article such as a feminine hygiene pad or component thereof is a lateral line lying in an x-y plane and equally dividing the length of the pad or the component when it is laid out flat on a horizontal surface. A lateral axis is perpendicular to a longitudinal axis.

“Longitudinal”—with respect to an absorbent article such as a feminine hygiene pad, or a component thereof, refers to a direction perpendicular to the lateral direction. A “length” dimension of any component or feature of the article is measured along the longitudinal direction from its forward extent to its rearward extent. When an article such as a feminine hygiene pad or component thereof is laid out flat on a horizontal surface, the “longitudinal” direction is perpendicular to the lateral direction relative the pad when it is worn, as defined above. With respect to an absorbent article, the “y-direction” is also the longitudinal direction.

The “longitudinal axis” of a feminine hygiene pad or component thereof is a longitudinal line lying in an x-y plane and equally dividing the width of the pad or component, when the pad is laid out flat on a horizontal surface. A longitudinal axis is perpendicular to a lateral axis.

“x-y plane,” with reference to an absorbent article, such as a feminine hygiene pad, or component thereof, when laid out flat on a horizontal surface, means any horizontal plane occupied by the horizontal surface or any layer of the article or component.

“z-direction,” with reference to an absorbent article, such as a feminine hygiene pad or component thereof, when laid out flat on a horizontal surface, is a direction perpendicular/orthogonal to the x-y plane.

The terms “top,” “bottom,” “upper,” “lower,” “over,” “under,” “beneath,” “superadjacent,” “subjacent,” and similar terms characterizing relative vertical positioning, when used herein to refer to layers, components or other features of an absorbent article such as a feminine hygiene pad, are relative the z-direction and are to be interpreted with respect to the pad as it would appear when laid out flat on a horizontal surface, with its wearer-facing surface oriented upward and outward-facing surface oriented downward.

With respect to an absorbent article such as a feminine hygiene pad, or a component or structure thereof, “wearer-facing” is a relative locational term referring to a feature of the component or structure that when in use that lies closer to the wearer than another feature of the component or structure. For example, a topsheet has a wearer-facing surface that lies closer to the wearer than the opposite, outward-facing surface of the topsheet.

With respect to an absorbent article such as a feminine hygiene pad, or a component or structure thereof, “outward-facing” is a relative locational term referring to a feature of the component or structure that when in use that lies farther from the wearer than another feature of the component or structure. For example, a topsheet has an outward-facing surface that lies farther from the wearer than the opposite, wearer-facing surface of the topsheet.

“Machine direction” or “MD” as used herein with respect to an absorbent article such as a feminine hygiene pad or component thereof, refers to a direction parallel to the flow of the article or component through processing/manufacturing equipment. With respect to manufacture of a web material, the “y-direction” is a direction parallel with the machine direction.

“Cross direction” or “CD” as used herein with respect to an absorbent article such as a feminine hygiene pad or component thereof, refers to a direction perpendicular/orthogonal to the machine direction. With respect to manufacture of a web material, the “x-direction” is a direction parallel with the cross direction.

“Predominant,” and forms thereof, when used to characterize a quantity of weight, volume, surface area, etc., of an absorbent article or component thereof, constituted by a composition, material, feature, etc., means that a majority of such weight, volume, surface area, etc., of the absorbent article or component thereof is constituted by the composition, material, feature, etc.

### General—Absorbent Article; Feminine Hygiene Pad

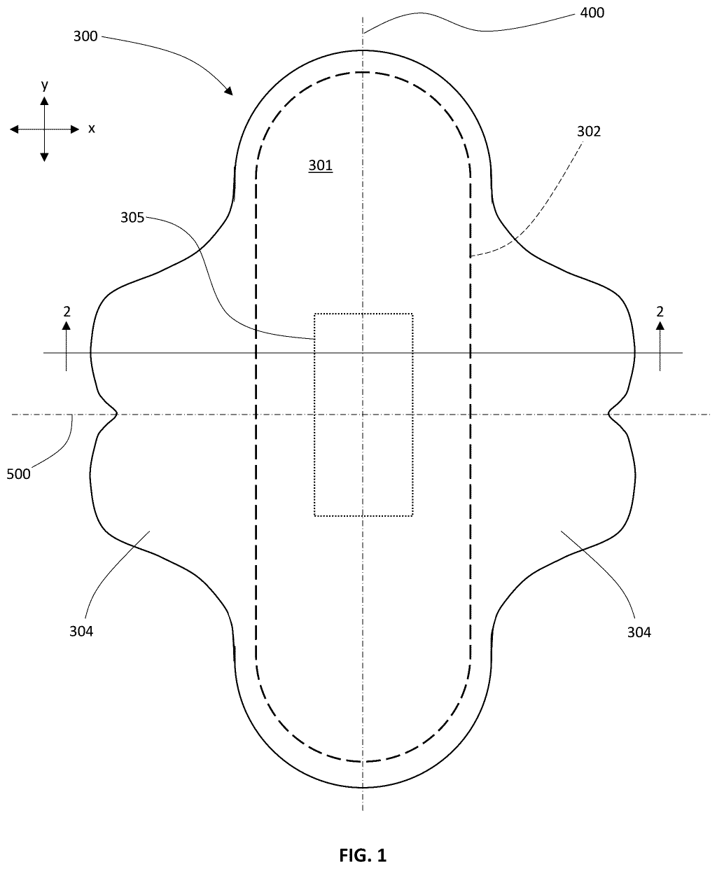

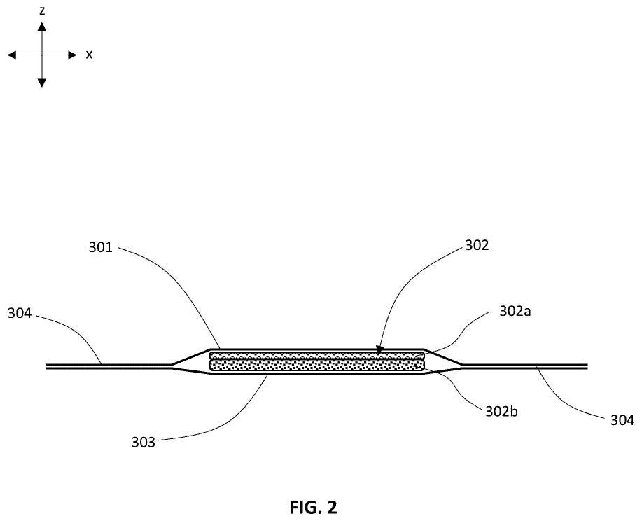

Referring to FIGS. 1 and 2, an absorbent article as contemplated herein, such as a feminine hygiene pad 300, will have a longitudinal axis 400 and a lateral axis 500, and include a wearer-facing surface and an opposing outward-facing surface. A liquid permeable topsheet 301 may form at least a portion of the wearer-facing surface and a liquid impermeable backsheet may form at least a portion of the outward-facing surface. An absorbent structure 302 is disposed between the topsheet and the backsheet. The absorbent structure 302 may include a fluid management layer 302a and a storage layer 302b. (A fluid management layer as described herein is sometimes known in the art as an “acquisition/distribution layer” “distribution layer” or “secondary topsheet”, whose purpose is to dissipate energy from a fluid gush to the extent needed, provide a temporary volume of space for discharged fluid to occupy during the time required for an underlying absorbent structure (storage layer) to imbibe and absorb the fluid, and to distribute the fluid across the absorbent structure to maximize effective use thereof.) Non-limiting examples of absorbent articles sharing these features include feminine hygiene pads (also known as “sanitary napkins”, “menstrual pads,” etc.), disposable incontinence pads, disposable incontinence underwear, disposable baby diapers and disposable baby/child training pants. Non-limiting examples of suitable absorbent structures/storage layers are described in co-pending U.S. Prov. App. Ser. No. 63/256,164, which is incorporated by reference herein. Non-limiting examples of suitable absorbent structures/storage layers are described in co-pending U.S. Prov. App. Ser. No. 63/316,097, which is incorporated by reference herein.

The topsheet 301 and the backsheet 303 may be joined together to form and define an outer periphery of the pad. The absorbent structure 302 and components or layers thereof (e.g., fluid management layer 302a and storage layer 302b) will be sized such that their outer perimeters are disposed laterally and longitudinally inboard of the outer periphery. Components or layers of the absorbent structure 302 may be dimensioned and shaped substantially similarly or identically to each other in the x-y directions, or they may have respective differing x-y dimensions and/or shapes. Individual layers may be manufactured to have a stadium shape as suggested in FIG. 1, or one or both may be manufactured to have any other suitable shape, such as an oval shape, rectangular shape, rounded rectangle shape, hourglass shape, peanut shape, etc. Shapes having concave profiles along the longitudinal edges (for example, an hourglass shape) may in some examples provide for enhanced comfort and/or conformity with the wearer's body.

The topsheet 301 may be joined to the backsheet 303 by any suitable attachment mechanism. The topsheet 301 and the backsheet 303 may be joined directly to each other in the article periphery, and may be indirectly joined together by directly joining them to the absorbent structure 302, the fluid management layer 302a and/or storage layer 302b, and/or additional layers disposed between the topsheet 301 and the backsheet 303. This indirect or direct joining may be accomplished by any suitable attachment mechanism known in the art. Non-limiting examples of attachment mechanisms may include e.g. fusion bonds, ultrasonic bonds, pressure bonds, adhesive bonds, or any suitable combination thereof.

Pad 300 may include a pair of oppositely disposed lateral extensions (sometimes called “wings”) 304 which do not include absorbent components. Wings 304 may be formed of or include lateral extensions of one or both of the topsheet 301 and the backsheet 303 materials. Wings 304 may also include deposits of an adhesive (not shown) on the outward-facing surfaces thereof. With this configuration, a user may appropriately locate and place pad 300 within the crotch region of her underpants, and wrap and fold wings 304 down, over and around the respective edges of the leg openings, and then adhere the wings 304 to the outside/underside of the underpants crotch region. So positioned, the wings 304 can help hold the pad in suitable position during wear/use, and help protect the underpants from soiling about the leg edges.

### Topsheet

### General

Generally, it is desirable that the topsheet 301 be compliant, soft feeling, and non-irritating to the wearer's skin. A suitable topsheet material will include a liquid pervious material that is disposed to the wearer-facing side of the article, in a position in which it will contact the body of the wearer. Preferably, the topsheet will be configured to permit discharged fluid to rapidly penetrate through it, and desirably, not readily allow fluid to move back up through the topsheet and contact the wearer's skin. The topsheet may also be adapted to bear and/or provide for transfer or migration of a selected lotion composition provided with the article, to the wearer's skin. The topsheet may include or be formed of a nonwoven material.

Nonwoven webs to be used as components of topsheets may be produced by any known procedure for making nonwoven webs, nonlimiting examples of which include spunbond processes, carding, wet-laying, air-laying, meltblowing processes, needle-punching, mechanical entangling, thermo-mechanical entangling, and hydroentangling.

Nonwoven materials suitable for use as a topsheet component material may include one stratum of accumulated fibers or may be laminate of multiple strata of accumulated fibers, which may include similar or different compositions (e.g., spunbond-meltblown laminate). In one specific example, the topsheet may be formed of a carded, air-through bonded nonwoven web material.

Topsheets contemplated herein do not include any predominant fraction of topsheet x-y surface area occupied by film. Some currently known topsheets for feminine hygiene pads include an apertured film, such as a hydroformed film or vacuum-formed film, alone or in combination with an adjacently-disposed nonwoven web material. The film may help to prevent liquids from resurfacing and contacting the wearer. The inventors have found, however, that a topsheet having the features described herein, for example, when combined with an appropriately composed and configured fluid management layer (for example as disclosed in in co-pending U.S. Prov. App. Ser. No. 63/256,164) can effectively prevent rewet to a comparable degree, or better, than pads having topsheets comprising film across a predominant portion of topsheet x-y surface area. Without intending to be bound by theory, it is believed that the careful selection of the fiber types in each of the strata in the fluid management layer, and the linear densities of the fiber types, can result in a desired combination of suitably low fluid acquisition time, and low rewet, overcoming the typical tradeoff in these conflicting objectives associated with prior nonwoven topsheets. The improved performance is evident from the new combination of the unique nonwoven topsheet with a fluid management layer of the present disclosure.

In addition to the features described herein, nonwoven web material used to form a topsheet may have any of the features, structures, components and/or compositions described in, for example, co-pending US provisional applications Ser. Nos. 63/256,164 and 63/316,097, the disclosures of which are incorporated herein by reference.

### Basis Weight

The topsheet nonwoven may be manufactured to a basis weight of at least about 15 gsm, more preferably at least about 40 gsm, or most preferably at least about 60 gsm, specifically reciting all values within these ranges and any ranges created thereby. In some examples, a nonwoven topsheet contemplated herein may be manufactured to have a basis weight of about 15 gsm to 80 gsm, more preferably about 20 gsm to 60 gsm, or most preferably about 20 gsm to 40 gsm, specifically reciting all values within these ranges and any ranges created thereby. In particular examples the topsheet nonwoven may be manufactured to a basis weight of about 18 gsm to 40 gsm, more preferably about 20 gsm to 30 gsm, even more preferably about 22 gsm to 26 gsm, specifically reciting all values within these ranges and any ranges created thereby. The range of desirable basis weight is influenced, at the lower end of the range, by the need for a level of web tensile strength needed for processing, and by consumer preferences for a level of opacity and substantiality of loft, feel and appearance. The range of desirable basis weight is influenced, at the upper end of the range, by the need for suitable rapid fluid acquisition and passage of fluid through the topsheet, and material cost concerns.

### Fiber Composition

Nonlimiting examples of constituent materials suitable for use in a topsheet nonwoven include fibrous materials made from natural fibers, e.g., cotton, including 100 percent organic cotton, modified natural fibers, semi-synthetic fibers (e.g., fibers spun from regenerated cellulose) synthetic fibers (e.g., fibers spun from polymer resin(s)), or combinations thereof. Synthetic fibers may include fibers spun from single polymers or blends of polymers. Synthetic fibers may include monocomponent fibers, bicomponent fibers or multicomponent fibers. (Herein, bi- or multicomponent fibers are fibers having cross sections divided into distinctly identifiable component sections each formed of a single polymer or single homogeneous polymer blend, distinct from that of the other section(s). Such fibers and processes for making them are known in the art. Examples of bicomponent fiber configurations with substantially round cross sections include side-by-side or “pie slice” configurations, eccentric sheath-core configurations and concentric sheath-core configurations.

Nonwoven topsheets contemplated herein may include fibers having myriad combinations of constituent components. For example, fibers may be spun from polymeric materials, such as polyethylene (PE) and/or polyethylene terephthalate (PET). Fibers may be spun in the form of bi-component fibers. In some examples, bi-component fibers may have a core component of a first polymer (for example, PET) in combination with another polymer as a sheath component, in a sheath-core bicomponent configuration. In more particular examples, PE may form the sheath component in combination with a PET core component. Fibers that include a PET component may be selected to help provide bulk and resilience and a resulting cushiony feel to the nonwoven web. Additionally, fibers that include a PET component, having comparatively greater resilience, help the web retain the area and dimensions of apertures created therethrough, if included.

Other polymeric materials may be included. For example, fibers spun of polypropylene, polyethylene, co-polyethylene terephthalate, co-polypropylene, and other thermoplastic resins may be included. It may be desired that the polymer with the lower melting temperature form the sheath component when sheath-core bi-component fibers are included. Additionally, without intending to be bound by theory, it is believed that the use of polyethylene terephthalate as a core component can help impart resilience to the topsheet.

Polyethylene, as a polymer component from which fibers may be spun, has a relatively lower melting temperature, and exhibits a relatively slick/silky surface feel as compared with other potentially useful polymers. PET has a relatively higher melting temperature, and exhibits relatively greater stiffness and resiliency. Accordingly, in some examples topsheet nonwoven fibers that are of a sheath-core bicomponent configuration may be desired, in which the sheath component is predominantly polyethylene and the core component is predominantly PET. The polyethylene is useful for imparting the fibers and thus the topsheet with a silky feel, and for enabling inter-fiber bonding via heat treatment that cause sheaths of adjacent/contacting fibers to melt and fuse at the lower melting temperature of the polyethylene, while the PET is useful for imparting resilience, and due to its higher melt temperature does not melt in a heat treatment process involving suitably controlled temperature(s). The inventors have found that a suitable weight ratio in such PE/PET sheath-core bicomponent fibers may be about 40:60 to about 60:40.

The constituent fibers may be staple fibers. The staple fibers may be carded, and consolidated to create a web having cohesiveness and tensile strength via a spunlacing process or other suitable process that integrates and/or entangles the fibers. In some examples the web may be calender bonded to impart additional consolidation and tensile strength. In other examples, however, calender bonding might be, preferably, foregone, because it can reduce web caliper and loft, reduce porosity, increase web stiffness, and adversely affect or reduce other softness attributes perceivable by consumers.

### Surface Treatment (Hydrophilicity/Hydrophobicity)

Depending upon the chemical composition thereof, surfaces of fibers will be, inherently, either hydrophilic or hydrophobic. For example, surfaces of fibers spun or otherwise formed from some types of polymers such as polyethylene and polypropylene will be, inherently, hydrophobic. In contrast, surfaces of other types of fibers such as fibers spun from regenerated cellulose (e.g., rayon, viscose, lyocell, etc.) are inherently hydrophilic. Surfaces of natural fibers may be inherently hydrophilic or hydrophobic, but this may depend upon the processing the fibers have undergone. For example, cotton fibers as harvested bear coatings of natural waxes and as such their surfaces are hydrophobic. After they have undergone processes including scouring and bleaching, however, the waxes will have been stripped away, rendering the fiber surfaces hydrophilic.

Manufacturers and/or suppliers of spun synthetic staple fibers currently apply coatings, in the form of surface finishing agents or processing aids, to the fibers, for purposes of providing lubricity in, e.g., carding processes. These surface finishing agents or processing aids may be formulated to be either hydrophobic or hydrophilic, and substantially durable for purposes herein, in that they will not dissolve in aqueous fluids over the ordinary duration of wear of an absorbent article. Thus, a manufacturer or supplier of spun synthetic staple fibers may offer fibers with either hydrophobic or hydrophilic surface finishes, and currently, several manufacturers in the nonwovens materials industry do this.

Noting that spun synthetic staple fibers may be obtained with either inherently hydrophobic, or inherently hydrophilic, surfaces, or obtained with surface finishes that render their surfaces hydrophilic or hydrophobic at the purchaser's option, it may be desirable to choose fibers with surfaces that are either hydrophilic (“hydrophilic fibers”) or hydrophobic (“hydrophobic fibers”),or, to choose a blend of fibers of both types.

In some examples it may be preferable that the fiber constituents of the topsheet be, by weight, predominantly, substantially, or entirely hydrophobic, or rendered hydrophobic via fiber surface finish. A topsheet formed of a nonwoven web with predominantly hydrophobic fiber constituents will be resistant to rewetting. On the other hand, if the sizes of the pores or inter-fiber voids within the fibrous structure of such nonwoven web are not sufficiently large, the topsheet may resist the passage of fluid from the wearing facing surface through to the absorbent core components of the article therebeneath, i.e., will not readily/rapidly acquire fluid, unless other features are included in combination, as described herein.

In other examples, fibers constituting portions, a majority (by surface area), or all, of the section of web material from which of the topsheet is formed, may be a blend of both hydrophobic fibers and hydrophilic fibers. In such examples, the hydrophilic fibers can serve to help wick fluid from the wearer-facing surface of the topsheet down to the absorbent core components beneath, while the hydrophobic fibers can serve to help the topsheet resist rewetting. The inventors have discovered that a successful balance may be struck for such examples.

Accordingly, in some examples the topsheet nonwoven may include a mix of hydrophobic and hydrophilic fibers. For example, the nonwoven may include at least about 40 percent, more preferably at least about 50 percent, or most preferably at least about 60 percent hydrophilic fibers by weight of the fibers, specifically including all values within these ranges and any ranges created thereby. In more particular examples, the nonwoven topsheet may comprise about 40 percent to 70 percent, more preferably about 45 percent to 68 percent, or most preferably from about 50 percent to 65 percent, by weight, hydrophilic fibers, specifically reciting all values within these ranges and any ranges created thereby. The topsheet nonwoven may include a blend of hydrophilic fibers and hydrophobic fibers in a weight ratio of hydrophilic fibers to hydrophobic fibers of 30:70 to 70:30, more preferably 35:65 to 65:35, and even more preferably 40:60 to 60:40. As noted above, the hydrophilicity of the hydrophilic fibers may be effected by application of a surface treatment composition.

Without intending to be bound by theory, it is believed that where a majority of the fibers are hydrophilic, fluid acquisition speed can be improved by combination with other features described herein, while not overly impacting rewet in a negative or unacceptably negative manner. Where less rewet is the goal, then the converse may be true. In this circumstance, a higher weight fraction of hydrophobic fibers may be desired.

### Linear Density

Fibers are typically manufactured, selected and purchased by linear density specification, expressed as denier or decitex. For fibers of a given polymer constitution, linear density correlates with fiber size/diameter.

In some examples, the fibers constituting the topsheet may selected to have an average linear density of about 1.0 to 3.0 denier, more preferably about 1.5 to 2.5 denier, and even more preferably about 1.8 to 2.2 denier, and all combinations of subranges within these ranges are contemplated herein. In other examples, the fibers constituting the topsheet may be selected to have an average linear density of about 3.0 to 5.0 denier, more preferably about 3.5 to 4.5 denier, and even more preferably about 3.8 to 4.2 denier, and all combinations of subranges within these ranges are contemplated herein.

Without intending to be bound by theory, it is believed that, for a nonwoven of particular selected basis weight as contemplated herein, inclusion of fibers having a linear density greater than about 5.0 denier may result in a topsheet that lacks, for some consumers, a sufficiently soft feel, since such relatively larger fibers would tend to be stiffer. Conversely, a selection of fibers having a linear density less than about 1.0 denier result in unduly small interstitial spaces/voids between and among the fibers, and make fluid acquisition and movement through the topsheet unacceptably difficult unless apertures are included. In any event, a suitable pattern of apertures may be imparted to the topsheet, to increase liquid permeability.

### Staple Fiber Length

Suitable fibers may be staple fibers having a length of at least about 30 mm, 40 mm, or 50 mm, up to about 55 mm, or about 30 to 55 mm, or about 35 to 52 mm, reciting for said range every 1 mm increment therein. In particular example, staple fibers may have a length of about 38 mm.

### Apertures

The inventors have found that, in topsheet nonwovens that are formed of fibers of relatively small size/linear density and/or fibers that are predominantly, substantially or entirely hydrophobic, acquisition speed may be substantially increased by forming a pattern of apertures through the web. Generally, the preferred apertures will have sizes that are substantially larger than the average pore/void size (size of inter-fiber spaces) within the nonwoven web.

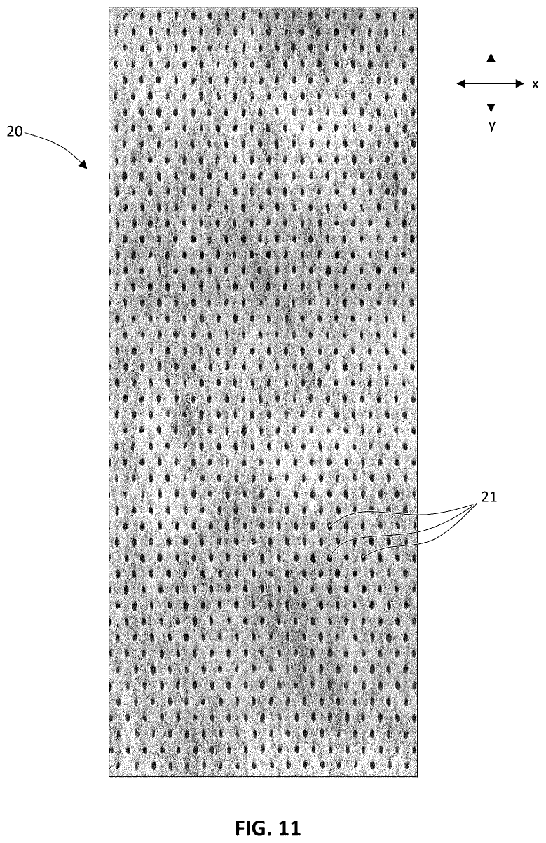

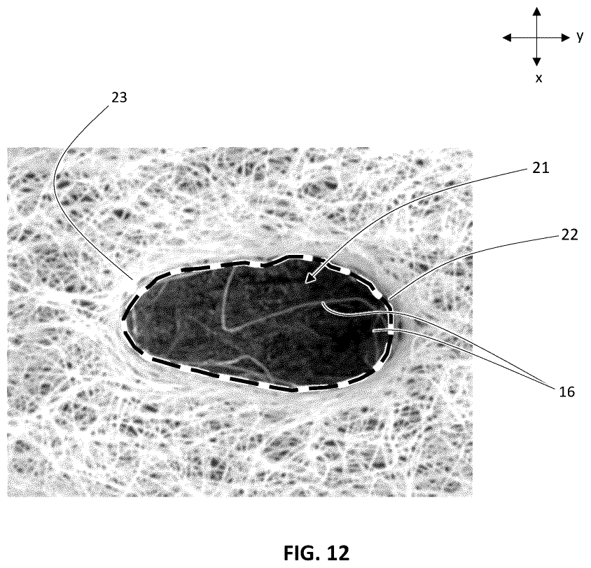

An example of a section of apertured topsheet nonwoven web material 20 having a pattern of apertures 21 therethrough is depicted in FIG. 11. A magnified image of example an aperture through a nonwoven web material is depicted in FIG. 12. Apertures are distinguishable from randomly-disposed pores or voids through the nonwoven web material, in that they are created by readily discernible displacements of fibers and/or fiber component material, along x-y directions, resulting in concentrated groups of densified material and/or displaced fibers that define the perimeter 22 of a z-direction hole/opening through the nonwoven web that is relatively larger than the randomly distributed pores or voids between and among the fibers constituting the nonwoven material.

Apertures may be created through the web via a process and equipment described herein and configured to impart an average x-y dimension aperture area of 0.1 mm² to 2.5 mm², preferably 0.3 mm² to 1.5 mm², and even more preferably from 0.3 mm² to 1.2 mm², and even more preferably 0.3 mm² to 0.5 mm²; and all combinations of subranges within these ranges are contemplated herein. Herein, the x-y dimension area of an aperture is defined by visually discernible inside edges of the densified zone 23 about the perimeter 22 of the aperture. Stray individual fibers that may have escaped the main structure and/or the densified zone about the perimeter, and cross into or through the main open area of the aperture (by way of illustrative example, stray individual fibers 16 shown in FIG. 12) are not considered subtractive from the aperture area for purposes herein. Further, without intending to be bound by theory, it is believed that the where the shapes of the apertures are too oblong or narrow, fluid acquisition speed may be negatively impacted. Accordingly, it may be desired that the apertures have a limited maximum average x-y direction aspect ratio (greatest dimension:smallest dimension in x-y directions). Thus, it may be desired that the average that the average aspect ratio of the apertures be about 2.5:1 to 1:2.5; more preferably 2:1 to 1:2; even more preferably from 1.5:1 to 1:1.5, still more preferably from 1.3:1 to 1:1.3, and most preferably from 1.2:1 to 1:1.2; all combinations of subranges within these ranges are contemplated herein. Further, it is preferable for purposes of retaining structural integrity of the web and shape integrity of the apertures, that the x-y plane shapes of the majority or all of the apertures in, at least, the region of interest 305 (“ROI,” defined below; see FIG. 1) if not the majority or entirety of the topsheet, be fundamentally rounded shapes (e.g., circular, oval, ovoid, elliptical, stadium, etc.), having no defined sharp corners. Accordingly, it may be desired that the pins on the roller used to create the apertures have radially outermost acting surfaces having shapes that do not define sharp corners, when viewed along a radially inward direction toward the axis of the roller.

Collectively, the aperture areas of all of the apertures in the portion of interest of the topsheet amount to an open x-y plane area (“open area”) in the topsheet nonwoven. In combination with a desired average aperture size, the inventors have identified a desired open area, in order to effectively mitigate potential obstacles to fluid acquisition that may result from constitution of fibers of finer denier and/or fibers that are predominantly hydrophobic. Accordingly, it may be desired that apertures, if included, collectively provide an open area of 1 percent to 25 percent, more preferably 1 percent to 20 percent, more preferably 3 percent to 15 percent, and even more preferably 4 percent to 10 percent; all combinations of subranges within these ranges are contemplated herein. It is preferred that such amount of open area be present in substantially the entirety of the portion of the topsheet overlying the fluid management layer and/or absorbent structure, or at least, in the region of interest 305 (“ROI”) defined below (and see FIG. 1). The lower limits of these ranges are imposed by the need for efficacy/performance; the apertures should provide at least a minimum amount of open area in order to be effective as may be included for the purposes described herein. The upper limits of these ranges are imposed by the need for consumer acceptance; if the open area is too great, consumers may perceive that the topsheet is fragile or of poor quality; and further, the topsheet becomes less effective at retaining fluid therebeneath, and at masking staining by absorbed fluid present in the absorbent components beneath the topsheet.

Referring to FIG. 1, for purposes contemplated herein, a region of interest 305 (“ROI”) is a rectangular section of the topsheet that is 60.0 mm long in the longitudinal direction and 30.0 mm wide in the lateral direction, and is centered at the longitudinal and lateral center, in an x-y plane, of the pad 300. The percent fraction open area of the ROI 305 is the fraction of the x-y area therewithin that is open therethrough in the z-direction, by the collective presence of the apertures therewithin. Expressed differently, the percent fraction open area within the ROI is the total open x-y area of the apertures within the ROI, divided by 1,800 mm², times 100%.

The percent fraction open area in the ROI may be obtained in some examples from the specifications given to or provided by the manufacturer of the topsheet nonwoven web material. Where this is unavailable, it may be measured via any suitable measurement technique that may applied, in a manner consistent with the description of the x-y dimension area of an aperture area and description of “open area,” above, which may include but is not limited to the Apertures Open Area Measurement Method set forth below.

### Bonding

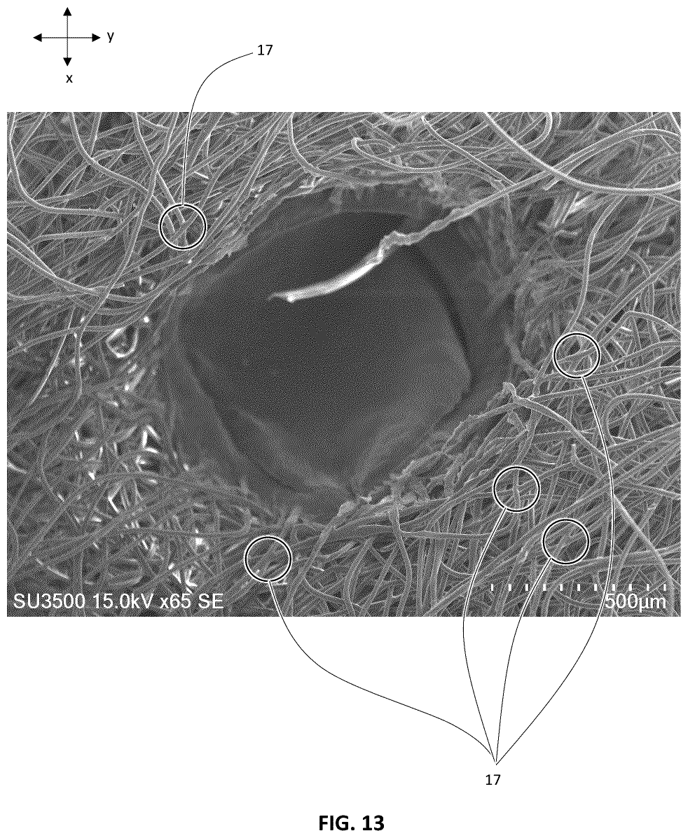

In some examples, it may be desirable that the fibers forming the topsheet nonwoven be bonded following the carding/fiber laydown process, to impart a fabric-like structure and tensile strength (in both the MD and the CD) needed for the web to substantially retain its structure in downstream/later processes, and in the form of a topsheet, during use by a user/wearer. As an alternative to other methods of bonding such as mechanical compression spot bonding (e.g., calender bonding) (with or without application of heating energy), adhesive bonding, etc., it has been found that bonding via air-through heating is effective for creating fiber-to-fiber bonds and imparting structural integrity to the web, while preserving inter-fiber pore/void size and loft, and imparting resiliency, to the nonwoven. Preserving resilient loft in this manner may be desired to mitigate potential loft/caliper reducing effects that may result from the aperturing process described herein, in which the web is compressed between aperturing rollers. In examples of suitable processes, air heated to the selected heating temperature is blown and/or drawn (via vacuum) through the carded fiber web as it is conveyed on a carrier belt along a machine direction, through an oven or heating chamber. When operating parameters including heating air temperature and velocity, and exposure time, are appropriately adjusted, a plurality of randomly distributed fiber-to-fiber bonds may be created within the fiber network, which impart structural integrity to the web. Examples of such fiber-to-fiber bonds 17 may be seen in FIG. 13. When constituent fibers are, for example, sheath-core bicomponent fibers in which the sheath component is a polymer having a melting temperature lower than that of the core component, the process may be configured such that fusion bonds form between sheaths of adjacent contacting fibers without complete melting and loss of structure of the sheaths, while the cores remain in place, un-melted. In such process, the bonds may be formed without application of z-direction compression or effects thereof, and thus, without associated loss of caliper of the web and reduction in size of the inter-fiber pores/voids.

### Aperturing Process, Resulting Features and Materials Selection

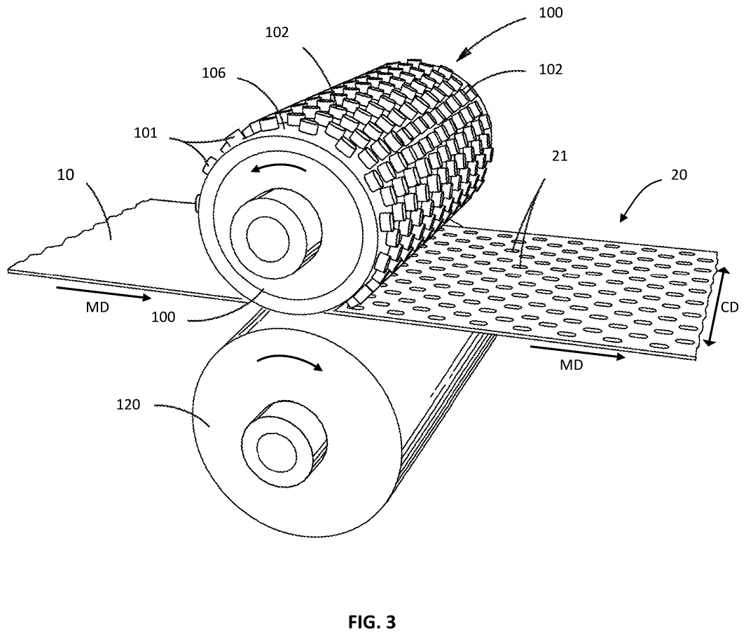

Referring to FIGS. 3 through 6B, generally cylindrical aperturing rollers, including aperturing roller 100 and opposing roller 120, operating upon a portion of a nonwoven web material are schematically depicted. Precursor web material 10 may be conveyed from an upstream supply 200, along a machine direction MD into a nip 110 between aperturing roller 100 and opposing roller 120, one or both of which are driven to rotate about axes that are parallel with each other and with a cross direction. An apertured nonwoven web material 20 exits the nip 110, have imparted therein an arrangement of apertures 21 of desired size, shape and configuration (as illustrated, in a nonlimiting example, in FIG. 11).

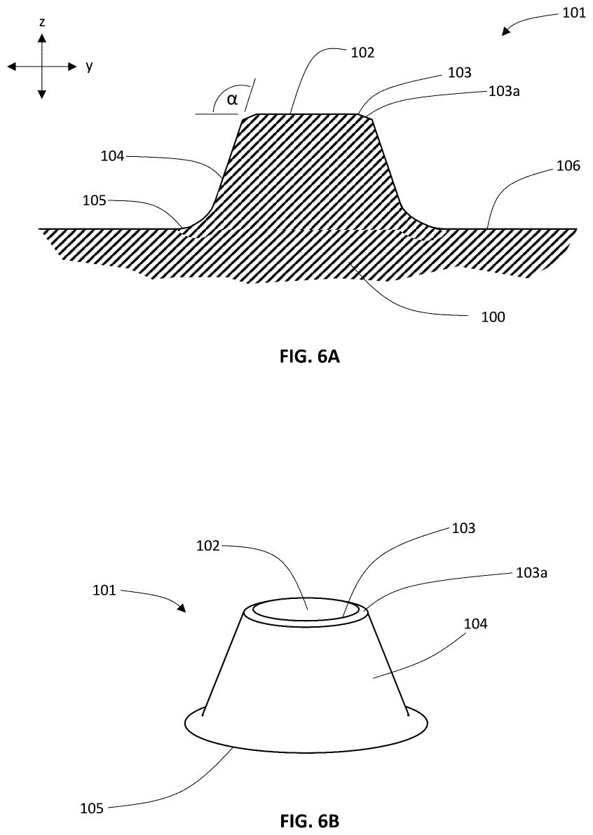

Aperturing roller 100 may have formed thereon and thereabout an arrangement of individual aperturing pins 101, which project radially outwardly from a base surface 106 of roller 100. Aperturing pins 101 have top surfaces 102 with surface areas that lie along an imaginary cylindrical shape. The top surface 102 of an aperturing pin 101 is, preferably, smooth and polished, with substantially no macroscopic cavities or irregularities therein. The areas of top surfaces 102 are defined and delimited by top surface perimeter edges 103. Preferably, at least following manufacturing of the roller, prior to wear of the pins from use thereof, top surface perimeter edge 103 is defined by an angular, not rounded, transition away from top surface 102. A small chamfer 103a about the top surface perimeter edge 103 may be included, as suggested in FIGS. 6A and 6B, to reduce stress concentration at the edge and chances of fracture thereabout. However, top surface perimeter edge 103 is not substantially radiused or rounded at the transition away from top surface 102. (The reason for this is explained below.) For purposes of mechanical structural integrity of the pin 101, preferably, pin base perimeter 105 will circumscribe a larger surface area at the base of the pin than that of top surface 102, and pin wall(s) 104 will taper inwardly towards each other from pin base perimeter 105 to pin top surface perimeter 103. In this regard, angle a between top surface 102 and pin wall(s) 104 will preferably be greater than 90 degrees. The pin may be concavely radiused or rounded about base perimeter 105, to reduce localized stress at the pin base perimeter during use of the roller.

Top surfaces 102 of aperturing pins 101 may be imparted with any desired shape(s) and size(s), generally corresponding with the desired x-y direction shape(s) and size(s) of the apertures to be formed in the subject nonwoven web material. Similarly, the aperturing pins 101 may be arranged on aperturing roller 100 in any desired pattern, generally corresponding to the desired x-y direction pattern of apertures to be formed in the subject nonwoven material.

For purposes of structural integrity, preferably, the aperturing pins 101 and portion of the aperturing roller forming the base surface 106 thereof are integral, formed of the same, contiguous mass of material. A predominant portion if not the entire aperturing roller may be integrally formed of the same, contiguous mass of material. Preferably the material of which pins 101 and base surface 106 are formed will be relatively hard and rigid, such as a suitable steel or alloy thereof. In some examples, pins 101 or at least top surfaces 102 may be imparted with a suitably selected non-stick or stick-resistant coating to avoid or reduce sticking of material to be melted and/or deformed in the nip.

In some examples, aperturing pins 101 may be formed by machining away or otherwise removing material from a solid cylindrical body, leaving behind material that defines and constitutes pins 101.

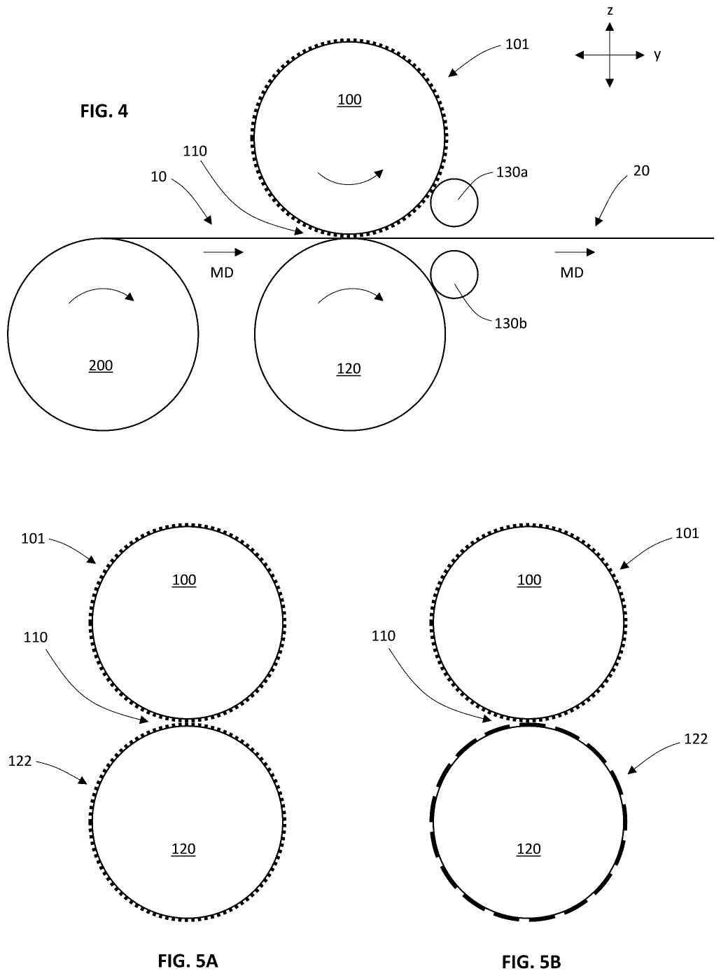

In some examples, as suggested in FIG. 4 opposing roller 120 may have a simple cylindrical outer surface that will meet all top surfaces 102 of aperturing pins 101 in the nip 110 between the rollers 100, 120. In other examples, opposing roller 120 may have thereon a formation of opposing structures 122 that effectively mirror, and meet the top surfaces 102, of aperturing pins 101 on aperturing roller 100, as suggested in FIG. 5A. In still other examples, as suggested in FIG. 5B, opposing roller 120 may have thereon a formation and arrangement of opposing structures 122 that are configured and arranged to meet desired subgrouping(s), but not all, of top surfaces 102 of aperturing pins 101 in the nip 110. In these latter examples, flexibility in providing a variety of selected differing configurations or patterns of apertures may be afforded, through manufacture and selected use of differing opposing rollers to be operated together with aperturing roller 100. Where only selective region aperturing of a nonwoven material is desired, these latter configurations may also enable extension of the useful life of the aperturing roller 100, since not all aperturing pins 101 need to be utilized at the same time.

As with aperturing roller 100 and aperturing pins 101, it is preferred that material forming the surface(s) of opposing roller 120 and/or any opposing structures 122 thereof be relatively hard and rigid. These surfaces, also, may be imparted with a suitable selected non-stick or stick-resistant coating.

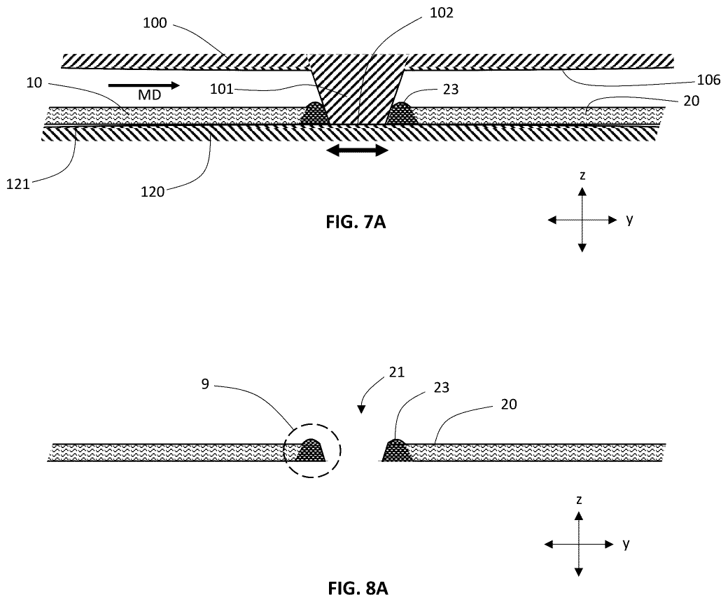

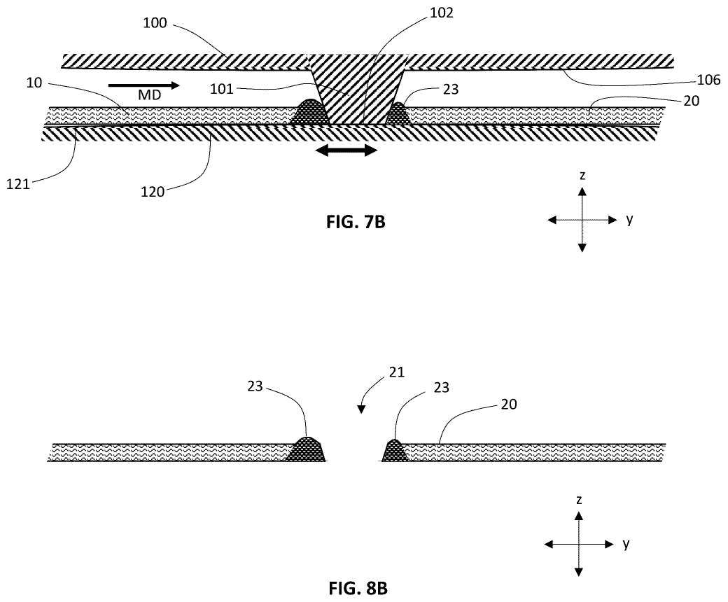

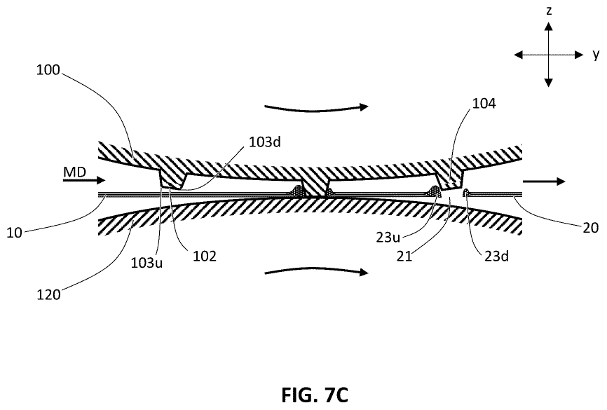

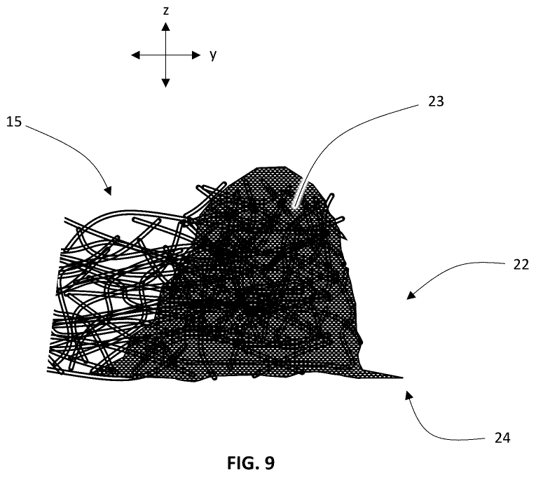

When configured for operation, aperturing roller 100 and opposing roller 120 are preferably arranged so that there is substantially or effectively no (zero) specified clearance in the nip 110, between the top surfaces 102 of the aperturing pins 101, and the opposing surface(s) of the opposing roller 120. Referring to FIG. 7, with such configuration, polymer material forming constituent fibers of a precursor nonwoven web material will be plastically deformed and expressed from the nipping regions between the top surfaces 102 of the aperturing pins and the opposing roller surface(s), as suggested by the double-headed arrow in FIG. 7, out to a zone beginning at and extending in an x-y direction beyond the perimeter edges 103 of top surfaces 102 of pins 101. Referring to FIGS. 7-10C, a densified aperture perimeter zone 23 results. Depending upon the composition(s) of the nonwoven fiber 15 constituents and the operating temperatures of the rollers, densified zone 23 may include densified polymer material that has been melted and then fused, unmelted but plasticly deformed and expressed fiber components, or a combination thereof. The formation of a densified zone 23 defining the aperture 21 stabilizes the size and shape of the aperture within the apertured nonwoven web 20.

As reflected in FIGS. 7B, 7C and 8B, in some circumstances, densified zones 23 in apertured web 20, resulting from the expressing of material from the nipping regions between the top surfaces 102 of the aperturing pins 101 and the opposing roller surface(s) 121, may be relatively larger and/or more dense in upstream regions 23u proximate the aperture perimeters 22 that were proximate the upstream edges 103u of the top surfaces 102, and relatively smaller and/or less dense in downstream regions proximate the aperture perimeters 22 opposite the upstream regions, proximate the downstream edges 103d of top surfaces 102, in the nip. Without intending to be bound by theory, it is believed, that when the system is configured for effectively zero specified clearance between the top surfaces 102 of pins 101, and the opposing roller surface(s) 121 as described herein, the downstream edges 103d and portions of top surfaces 102 will reach closest proximity or contact with the opposing roller surface(s) 121 first in time, and as the rollers rotate, closest proximity or contact between the top surfaces 102 of the pins will progress in rolling fashion from the downstream edges 103d toward the upstream edges 103u of the top surfaces 102 of the pins. As a result, the system will tend to express material of the precursor web material 10 as it passes through the nip, more in an upstream direction than a downstream direction, relative the web material 10. (Herein, “upstream” refers to a direction opposite the machine direction MD of web travel through the nip, and “downstream” refers to a direction the same as the direction MD of web travel through the nip.)

Either or both of aperturing roller 100 and opposing roller 120 may be heated to a temperature at or above the melt temperature of one or more of the polymer components of web fiber constituents, to facilitate such deformation and/or melting and fusing.

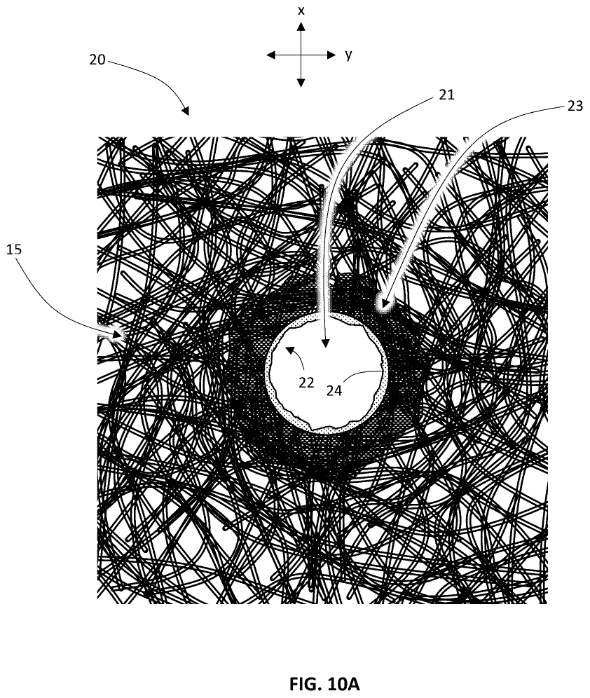

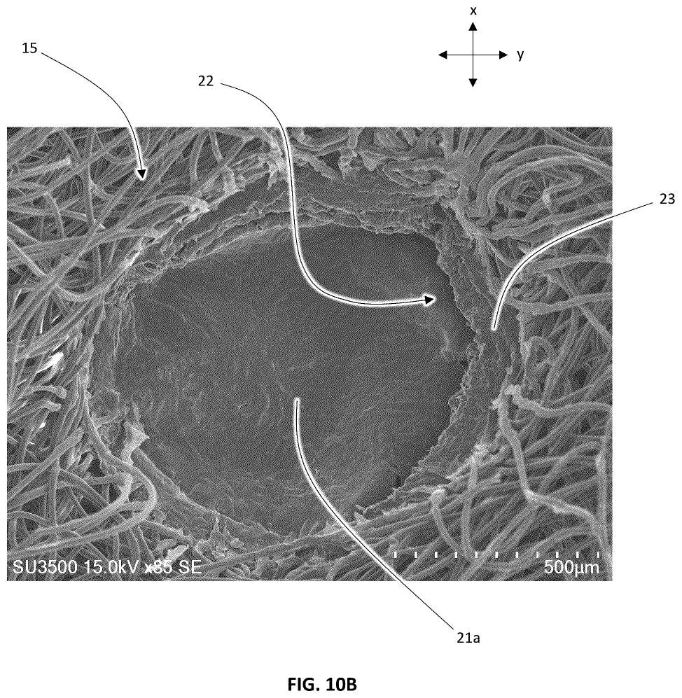

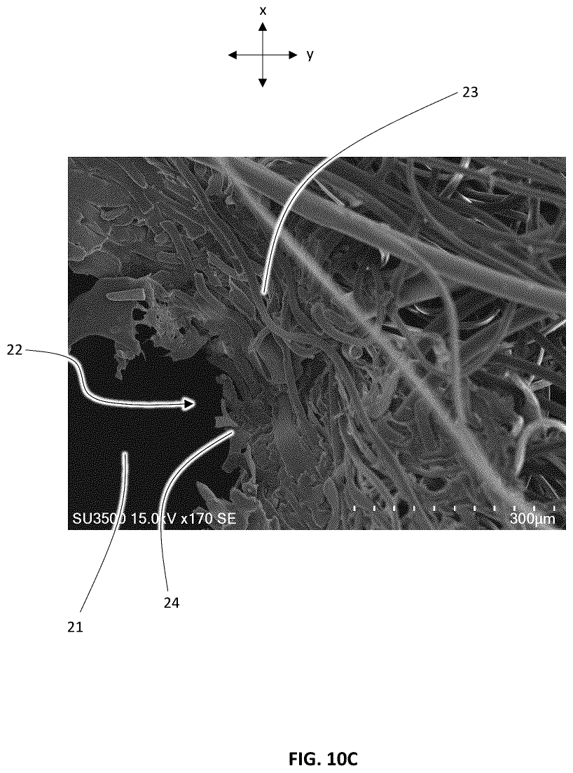

Even when the rollers are configured with substantially or effectively zero specified clearance in the nip 110, it may be difficult to cause expression of the entirely of the fiber component material(s) caught in the nipping regions between the pin top surfaces 102 and the opposing roller surface(s) 121. This can be the result of microscopic surface imperfections in the pin top surfaces 102 and/or opposing roller surfaces 121 and/or some compliance intentionally provided in the respective roller structures or roller carrying and/or driving mechanisms and structures, where an unfeasibly high amount of pressure in the nip would be required to express all material from the nipping regions between the tops of the aperturing pins and the opposing roller surface(s). This latter circumstance would be present in examples in which fiber components are present in the subject nonwoven web which are brittle or not substantially ductile, and which, as a result, are crushed and flattened beneath the pins but not expressed. Such examples may include nonwoven web materials constituted in part by fibers composed of non-thermoplastic materials, such as regenerated cellulose (e.g., rayon, viscose, lyocell, etc.). In some examples it may be desired to include with one or both of the rollers, or roller carrying and/or driving mechanisms and structures, features that provide limited compliance at a given pressure, i.e. allow the rollers to separate to a limited extent at the nip 110 at a given nip pressure, to delimit maximum pressure in the nip that is exerted on the pin surfaces 102, to reduce roller wear and chances of pin failure. As a result, a very thin/low caliper film 21a (shown in FIG. 10B, in one example) of unexpressed polymer and/or other fiber component material may remain within the apertures 21, as the apertured web 20 exits the nip 110. When component materials of the web constituent fibers are appropriately selected as described below, however, this thin film 21a will readily break away and out of the web, leaving behind an open aperture 21. The resulting apertures 21 may have about their perimeters 22 a fracture edge 24, which will be a remainder of the thin film 21a following break-away of the majority thereof. When the perimeter edges 103 of the pin top surfaces 102 are sharply defined, as described above, the amount of this film forming a fracture edge 24 will be minimized, i.e., the fracturing away of the film will desirably occur very close to the densified zone 23 and aperture perimeter 22.

In some circumstances the unexpressed film material may fracture away from the apertures 21 and adhere to either or both of the pin top surfaces 102 and the opposing roller surface(s) 106 as the nonwoven web 20 exits the nip 110. To address this, the system may be provided with online roller cleaning equipment 130a and/or 130b. Such roller cleaning equipment may consist of or include one or more scraper blades, brushes (which may themselves be configured to operate on counter-rotating rollers), a system of one or more pressurized air knives, water jets, or any combination thereof, or any other suitable roller cleaning equipment.

If component materials of nonwoven constituent fibers are suitably selected, the process and mechanism described above can be effective at imparting a nonwoven web with apertures at relatively high throughput rates, contributing to manufacturing efficiency.

As described herein, in some examples, a desired topsheet nonwoven web material may be constituted partially or entirely of bicomponent fibers, having first and second polymer components. In such examples, it may be desired that the respective polymer components have differing melt temperatures. When the melt temperatures of the respective components differ sufficiently, it is possible to operate the web aperturing system described above wherein one or both of the rollers 100, 120 is/are heated to a temperature(s) sufficient to cause one of the polymer components to melt, but not the other. Doing this causes the polymer component with the lower melt temperature to melt and readily flow out from beneath the aperturing pin top surfaces 102, beyond top surface perimeter edges 103. At the same time, the polymer component with the higher melt temperature will not melt, and is forced not only to plastically deform but also to fracture, as it is being expressed from beneath the pin top surfaces 102 in the nip 110. The thin film of unexpressed material that may be left behind as the web exits the nip 110 will be largely constituted by the component with the higher melting temperature—which will be, desirably, fractured into pieces which will easily fall or be drawn out of the apertures.

To achieve or enhance this effect, it may be desired that the first and second polymer components of the bicomponent fiber constituent have a difference in melt temperatures of at least about 44° C., more preferably at least about 72° C., and even more preferably at least about 100° C. This provides the operator with a broad range of temperatures to which it may heat one or both of the aperturing rollers, to cause melting of a first polymer component with a lower melt temperature, while avoiding melting of a second polymer component with a higher melt temperature.

In some examples such as those described herein, a suitable topsheet nonwoven may be constituted of sheath-core bicomponent fibers, in which polyethylene terephthalate (PET) constitutes the core component and polyethylene (PE) constitutes the sheath component. PET has a melt temperature of about 264° C., which is relatively high among potential thermoplastic polymer components deemed suitable for spinning fibers useful for purposes contemplated herein. This relatively high melt temperature leaves considerable breadth for selection of a second suitable polymer, since most currently known, suitable thermoplastic polymers suitable for spinning fibers useful for purposes herein have melt temperatures considerably lower than 264° C. Additionally, PET is relatively brittle, tending to fracture more than other suitable polymers, rather than plastically deform in a ductile manner, under heavy pressure, which is desirable for reasons described above. In contrast, PE has a melt temperate of about 110 to 130° C. (depending on specific form), and above the melt temperature will readily flow. This makes it suitable as a nonwoven constituent fiber component for purposes described above—including formation of a stable densified zone 23 surrounding apertures, that contribute to making the apertures and configuration/pattern thereof stable within the nonwoven in downstream processing and converting operations. PE also imparts other desirable characteristics to the nonwoven, as described above.

It is contemplated that the process described above may be applied simultaneously to two distinct nonwoven web materials together. The two distinct nonwoven web materials 10 may be conveyed together through a nip 110 between an aperturing roller 101 and an opposing roller 120, as described above. As the materials exit the nip, they will each have apertures 21 that are aligned along the z-direction. The respective nonwoven web materials will be bonded together to some extent, at the densified zones 23 surrounding the respective, aligned apertures 21, by thermoplastic fiber component material that has been melted and expressed from beneath the aperturing pins 101 in the nip, and then fused. For example, a topsheet nonwoven web material and a fluid management layer nonwoven material, as described herein or in references incorporated by reference herein, may be brought together and apertured in the manner described above. The fluid management layer may be constituted as described in, for example, U.S. Prov. App. Ser. No. 63/316,097. In some examples the fluid management layer may comprise any combination of monocomponent fibers, hollow monocomponent fibers, bicomponent fibers, cellulosic fibers, and regenerated cellulose fibers. The bicomponent fibers may spun to have a sheath-core configuration including a PET core component. The sheath component may be PE. The hollow monocomponent fibers may be spun of PET.

As noted, aperturing pins 101 can be formed with top surfaces 102 having a variety of surface areas and shapes, corresponding to the area and shape of the apertures one wishes to impart to the subject nonwoven web. It will be appreciated that a substantially circular-shaped aperture 21 in a web may be most efficient, per unit surface area, for providing a fluid passageway. Similarly, shapes that have aspect ratios (of machine direction dimension to cross-direction dimension) approaching 1:1 are relatively efficient, as compared to shapes having aspect ratios in which either dimension is substantially larger or smaller than the other. The process and equipment described herein may be configured to efficiently impart a pattern of apertures to a nonwoven web, wherein the apertures in the pattern have an average aspect ratio of from 2.5:1 to 1:2.5; more preferably 2:1 to 1:2; even more preferably from 1.5:1 to 1:1.5, still more preferably from 1.3:1 to 1:1.3, and most preferably from 1.2:1 to 1:1.2.

The process described above does not require that the web be substantially stretched in the machine or cross directions in a subsequent step, to open the apertures, following its exit from the nip.

### Test and Measurement Methods

### Caliper

The caliper, or thickness, of a test specimen is measured as the distance between a reference platform on which the specimen rests and a pressure foot that exerts a specified amount of pressure onto the specimen over a specified amount of time. All measurements are performed in a laboratory maintained at 23° C.±2 C.° and 50%±2% relative humidity and test specimens are conditioned in this environment for at least 2 hours prior to testing.

Caliper is measured with a manually-operated micrometer equipped with a pressure foot capable of exerting a steady pressure of 0.50 kPa±0.01 kPa onto the test specimen. The manually-operated micrometer is a dead-weight type instrument with readings accurate to 0.01 mm. A suitable instrument is Mitutoyo Series 543 ID-C Digimatic, available from VWR International, or equivalent. The pressure foot is a flat ground circular movable face with a diameter that is smaller than the test specimen and capable of exerting the required pressure. A suitable pressure foot has a diameter of 25.4 mm, however a smaller or larger foot can be used depending on the size of the specimen being measured. The test specimen is supported by a horizontal flat reference platform that is larger than and parallel to the surface of the pressure foot. The system is calibrated and operated per the manufacturer's instructions.

Obtain a test specimen by removing it from an absorbent article, if necessary. When excising the test specimen from an absorbent article, use care to not impart any contamination or distortion to the test specimen layer during the process. The test specimen is obtained from an area free of folds or wrinkles, and it must be larger than the pressure foot.

To measure caliper, first zero the micrometer against the horizontal flat reference platform. Place the test specimen on the platform with the test location centered below the pressure foot. Gently lower the pressure foot with a descent rate of 3.0 mm±1.0 mm per second until the full pressure is exerted onto the test specimen. Wait 5 seconds and then record the caliper of the test specimen to the nearest 0.001 mm. In like fashion, repeat for a total of ten replicate test specimens. Calculate the arithmetic mean for all caliper measurements and report as Caliper to the nearest 0.001 mm.

### Basis Weight

The basis weight of a sample of sheet or web material is the mass (in grams) per unit area (in square meters) of a single layer of the material. If it is not otherwise known or available, basis weight may be measured using EDANA compendial method NWSP 130.1. The mass of the test sample is cut to a known area, and the mass of the sample is determined using an analytical balance accurate to 0.0001 grams. All measurements are performed in a laboratory maintained at 23° C.±2 C.° and 50%±2% relative humidity and test samples are conditioned in this environment for at least 2 hours prior to testing.

Measurements are made on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained. The test specimen must be as large as possible so that any inherent material variability is accounted for.

Measure the dimensions of the single layer test specimen using a calibrated steel metal ruler traceable to NIST, or equivalent. Calculate the Area of the test specimen and record to the nearest 0.0001 square meter. Use an analytical balance to obtain the Mass of the test specimen and record to the nearest 0.0001 gram. Calculate Basis Weight by dividing Mass (in grams) by Area (in square meters) and record to the nearest 0.01 grams per square meter (gsm). In like fashion, repeat for a total of ten replicate test specimens. Calculate the arithmetic mean for Basis Weight and report to the nearest 0.01 grams/square meter.

### Material Compositional Analysis

If the information is not otherwise available, the quantitative chemical composition of a test specimen comprising a mixture of fiber types is determined using ISO 1833-1. All measurements are performed in a laboratory maintained at 23° C.±2 C.° and 50%±2% relative humidity.

Analysis is performed on test samples taken from rolls or sheets of the raw material, or test samples obtained from a material layer removed from an absorbent article. When excising the material layer from an absorbent article, use care to not impart any contamination or distortion to the layer during the process. The excised layer should be free from residual adhesive. To ensure that all adhesive is removed, soak the layer in a suitable solvent that will dissolve the adhesive without adversely affecting the material itself. One such solvent is THF (tetrahydrofuran, CAS 109-99-9, for general use, available from any convenient source). After the solvent soak, the material layer is allowed to thoroughly air dry in such a way that prevents undue stretching or other deformation of the material. After the material has dried, a test specimen is obtained and tested as per ISO 1833-1 to quantitatively determine its chemical composition.

### Average Fiber Decitex (dtex) or Denier

Textile webs (e.g., woven, nonwoven, airlaid) are comprised of individual fibers of material. Fibers are characterized in one respect, by their linear mass density, reported in units of denier, or units of decitex. The decitex value is the mass in grams of a fiber present in 10,000 meters of that fiber. The denier value is the mass in grams of a fiber present in 9,000 meters of that fiber. The average decitex or denier value of the fibers within a web of material is often reported by manufacturers as part of a specification. If the average decitex or denier value of the fiber is not otherwise known or available, it can be calculated by measuring the cross-sectional area of the fiber via a suitable microscopy technique such as scanning electron microscopy (SEM), determining the composition of the fiber with suitable techniques such as FT-IR (Fourier Transform Infrared) spectroscopy and/or DSC (Dynamic Scanning calorimetry), and then using a literature value for density of the composition to calculate the mass in grams of the fiber present in 10,000 meters of the fiber (for decitex), or in 9,000 meters of the fiber (for denier).

All testing is performed in a room maintained at a temperature of 23° C.±2.0° C. and a relative humidity of 50%±2% and samples are conditioned under the same environmental conditions for at least 2 hours prior to testing.

If necessary, a representative sample of web material of interest can be excised from an absorbent article. In this case, the web material is removed so as not to stretch, distort, or contaminate the sample.

SEM images are obtained and analyzed as follows to determine the cross-sectional area of a fiber. To analyze the cross section of a sample of web material, a test specimen is prepared as follows. Cut a specimen from the web that is approximately 1.5 cm (height) by 2.5 cm (length) and free from folds or wrinkles. Submerge the specimen in liquid nitrogen and fracture an edge along the specimen's length with a razor blade (VWR Single Edge Industrial Razor blade No. 9, surgical carbon steel). Sputter coat the specimen with gold and then adhere it to an SEM mount using double-sided conductive tape (Cu, 3M available from electron microscopy sciences). The specimen is oriented such that the cross section is as perpendicular as possible to the detector to minimize any oblique distortion in the measured cross sections. An SEM image is obtained at a resolution sufficient to clearly elucidate the cross sections of the fibers present in the specimen. Fiber cross sections may vary in shape, and some fibers may consist of a plurality of individual filaments. Regardless, the area of each of the fiber cross sections is determined (for example, using diameters for round fibers, major and minor axes for elliptical fibers, and image analysis for more complicated shapes). If fiber cross sections indicate inhomogeneous cross-sectional composition, the area of each recognizable component is recorded and dtex contributions are calculated for each component and subsequently summed. For example, if the fiber is bi-component, the cross-sectional area is measured separately for the core and sheath, and dtex contribution from core and sheath are each calculated and summed. If the fiber is hollow, the cross-sectional area excludes the inner portion of the fiber comprised of air, which does not appreciably contribute to fiber dtex. Altogether, at least 100 such measurements of cross-sectional area are made for each fiber type present in the specimen, and the arithmetic mean of the cross-sectional area aₖ for each are recorded in units of micrometers squared (μm²) to the nearest 0.1 μm².

Fiber composition is determined using common characterization techniques such as FTIR spectroscopy. For more complicated fiber compositions (such as polypropylene core/polyethylene sheath bi-component fibers), a combination of common techniques (e.g. FTIR spectroscopy and DSC) may be required to fully characterize the fiber composition. Repeat this process for each fiber type present in the web material.

The average decitex dₖ value for each fiber type in the web material is calculated as follows:

dₖ=10 000 m×aₖ×ρₖ×10⁻⁶

where dₖ is in units of grams (per calculated 10,000 meter length), aₖ is in units of μm², and ρₖ is in units of grams per cubic centimeter (g/cm³). Average decitex is reported to the nearest 0.1 g (per calculated 10,000 meter length) along with the fiber type (e.g. polypropylene (PP), PET, cellulose, PP/PET bicomponent). The average denier value for each fiber type in the web material is its decitex dₖ value×0.9.

### Apertures Percent Open Area Measurement Method

Percent open area is measured on images, of an apertured topsheet test specimen, acquired using a flatbed scanner. The scanner is capable of scanning in reflectance mode at a resolution of 2400 dpi and 8 bit grayscale. A suitable scanner is an Epson Perfection V750 Pro from Epson America Inc. (Long Beach, California, USA) or one having substantially similar functionality. The scanner is interfaced with a computer running an image analysis program. A suitable program is ImageJ v. 1.47 (National Institute of Health, USA), or one having substantially similar functionality. The specimen images are distance calibrated against an acquired image of a ruler certified by NIST. To enable maximum contrast, the specimen is backed with an opaque, background sheet of uniformly black color, prior to acquiring the image. All measurement is performed in a conditioned room maintained at about 23±2° C. and about 50±2% relative humidity.

The measurement specimens are prepared as follows.

Obtain the required number of samples of the absorbent article of interest. To obtain a measurement specimen, tape the sample absorbent article about its periphery (i.e., do not tape over regions underlaid by the fluid management layer), wearer-facing side up, in a flat configuration, to a horizontal flat work surface. Any elastic materials included (e.g., in leg cuffs), if present, may be cut to facilitate laying the article out flat. The outer boundary of the region of the apertured topsheet overlying the fluid acquisition layer of the article is identified and marked. Now cut through the topsheet and any adhered underlying layers, about and through this marked outer boundary with a new razor blade or other comparable new, sharp, cutting implement. From this cut out portion, the test specimen of the apertured topsheet is then carefully separated and removed from the underlying layer(s) such that its longitudinal and lateral dimensions are not changed, to avoid distortion of the apertures. If the topsheet is adhered via an adhesive to an underlying layer, before attempting separation apply any solvent suitable for dissolving the adhesive and allowing easy separation of the topsheet from underlying layer(s) without dissolving the polymer material(s) of fibers constituting the topsheet nonwoven web material. (In many examples, tetrahydrofuran (THF) can be a suitable solvent for this purpose. It is not a concern if the solvent dissolves applied surface finish coatings on the fibers, as long as it does not dissolve the polymer(s) constituting the fibers themselves.) Once the cut-out portion of the topsheet constituting the measurement specimen is removed, identify the wearer-facing side thereof. Five replicate measurement specimens obtained from five samples of the absorbent articles of interest, are prepared for measurement. The specimens are conditioned at about 23° C.±2 C.° and about 50%±2% relative humidity for 2 hours prior to imaging.

Images are obtained as follows.

The ruler is placed on the scanner bed such that it is oriented parallel to the sides of the scanner glass. An image of the ruler (the calibration image) is acquired in reflectance mode at a resolution of 2400 dpi (approximately 94 pixels per mm) and in 8-bit grayscale. The calibration image is saved as an uncompressed TIFF format file. After obtaining the calibration image, the ruler is removed from the scanner glass and all specimens are scanned under the following scanning conditions.

A measurement specimen is placed onto the center of the scanner bed, lying flat, with the body-facing surface of the specimen facing the scanner's glass surface. The corners and edges of the specimen are secured such that its original longitudinal and lateral dimensions, as on the article prior to removal, are retained. The specimen is oriented such that the long axis and short axis thereof are aligned parallel with and perpendicular to the sides of the scanner's glass surface, respectively. The black background is placed on top of the specimen, the scanner lid is closed, and a scanned image of the entire specimen is acquired with the same settings as used for the calibration image. The specimen image is saved as an uncompressed TIFF format file. The remaining four replicate specimens are scanned and saved in like manner.

The specimen image is analyzed as follows. Open the calibration image file in the image analysis program, and calibrate the image resolution using the imaged ruler to determine the number of pixels per millimeter. Now open the specimen image in the image analysis program, and set the distance scale using the image resolution determined from the calibration image. Now identify a rectangular section (region of interest, or “ROI”) longitudinally and laterally centered on the specimen, having a longitudinal dimension along the longitudinal axis of 60.0 mm and a lateral dimension of 30.0 mm, and visually inspect the images of the apertures present within the ROI. Now using the software tools, manually outline each of the apertures within the ROI (and any partial portions thereof at the edges of the ROI). The appropriate outlines will be drawn along visually discernible inside edges of the concentrations of displaced fibers 503 about the perimeters of the apertures. Stray individual fibers that may have escaped the main structure and/or the concentrations of displaced fibers about the perimeter, and cross into or through the main open area of the aperture (by way of illustrative example, stray individual fibers 504 shown in FIG. 5) are not considered subtractive from the aperture area for purposes herein.) Then use the software to measure the area within each discrete aperture outline (whole and partial) within the ROI and record each to the nearest 0.01 mm², and calculate the sum total thereof. The area of each discrete aperture is defined as the x-y surface area within the visually discernable outline of the open region, created by mechanical penetration of the web and x-y direction displacement of fibers in an aperturing process, that creates the apertures through the web. (For example, refer to FIG. 5 where discrete aperture area 501 and visually discernable boundary 502 are depicted. The dark area of the depicted aperture is an image of black construction paper used as a backing to the specimen of which this particular image was made.) The sum of the areas of all of the apertures within the ROI is recorded as Aperture Area to the nearest 0.01 mm². Now divide the Aperture Area by the ROI Area (1,800 mm²), then multiply by 100 and record as Open Area to the nearest 0.1%.

In like manner, repeat the entire procedure for the remaining four replicate specimen images. Calculate the arithmetic mean of Open Area across all five replicate specimens and report as Average Open Area to the nearest 0.1%.

### Non-Limiting Examples Contemplated Herein

In view of the foregoing description, the following non-limiting examples of combinations of features are contemplated herein. Where feasible, other features described herein may be included as well.
1. A single step method for imparting a pattern of apertures (21) in a fibrous nonwoven web (10) constituted predominantly of staple fibers, comprising the steps of:
   providing a pinned roller (100) having a first axis and bearing a pattern of aperturing pins (101) extending radially outwardly therefrom, wherein the aperturing pins have radially outermost top surfaces (102) which are substantially smooth and lie along a cylindrical shape profile about the first axis, the top surfaces having perimeter edges (103) thereabout that define surface areas of the top surfaces;
   providing an opposing roller (120) having a second axis;
   arranging the opposing roller and the pinned roller so as to form a nip (110) therebetween, wherein the first and second axes are substantially parallel, and wherein the nip has effectively zero clearance between the top surfaces of the aperturing pins and one or more outermost surfaces of the opposing roller,
   wherein the opposing roller comprises one or more radially outermost surfaces (121) that is/are cylindrical about the second axis, and disposed on the opposing roller so as to opposingly contact the top surfaces of the aperturing pins in the nip;
   providing heating energy to one or both of the pinned roller and the opposing roller;
   rotating the pinned and opposing rollers, and conveying the fibrous nonwoven web through the nip,
   whereby material(s) of which fibers (15) of the nonwoven web are constituted is/are at least partially melted and expressed from nip regions between the top surfaces of the pins and the opposing roller when the pins meet the opposing roller in the nip (110), outwardly towards the perimeter edges (103) of the pin top surfaces, and
   whereby expressed fiber material accumulates to form densified zones (23) about the perimeter edges (103) of the top surfaces (102) of the pins (101), that remain with the nonwoven web as it exits the nip;
   whereby a pattern of open apertures is imparted to the web, the pattern of apertures corresponding to the pattern of aperturing pins, thereby providing an apertured nonwoven web (20).
2. The method of example 1 wherein the perimeter edges (103) of the outermost top surfaces (102) of the aperturing pins (101) define shapes having an average aspect ratio of from 2.5:1 to 1:2.5; more preferably 2:1 to 1:2; even more preferably from 1.5:1 to 1:1.5, still more preferably from 1.3:1 to 1:1.3, and most preferably from 1.2:1 to 1:1.2.
3. The method of example 1 further comprising the step of air-through bonding the fibrous nonwoven web (10) or apertured nonwoven web (20), either prior to or subsequent to the step of conveying the fibrous nonwoven web (10) through the nip.
4. An apertured nonwoven web (20) comprising staple fibers, having a pattern of apertures (21) therethrough, wherein each of a plurality of the apertures is surrounded by a densified agglomeration of material(s) (23) of which fibers of the nonwoven web are composed, the materials having been plastically deformed and/or fused via z-application of localized z-direction direction compression and optionally, application of heat.
5. The apertured nonwoven web of example 4, wherein the densified agglomeration of materials (23) is relatively larger and/or more dense in a first region (23u) proximate a first portion of a perimeter (22) said each of said plurality of apertures, and relatively smaller and/or less dense in a second region (23d) proximate a second portion of the perimeter (22) of said each of said plurality of apertures, wherein the first portion of the perimeter is oppositely disposed of the second portion of the perimeter.
6. The apertured nonwoven web of example 5 wherein the first portion of the perimeter is disposed on an upstream side of said each of said plurality of apertures, and the second portion of the perimeter is disposed on a downstream side of said each of said plurality of apertures, wherein “upstream” and “downstream” are relative a machine direction along which the apertured nonwoven web was passed through a nip between a pair of aperturing rollers.
7. The apertured nonwoven web of any of examples 4-6, comprising a plurality of randomly distributed fiber-to-fiber bonds (17) therewithin, the fiber-to-fiber bonds not exhibiting effects of z-direction compression in the formation thereof.
8. The apertured nonwoven web of any of examples 4-7, wherein the apertures in the pattern have an average aspect ratio of from 2.5:1 to 1:2.5; more preferably 2:1 to 1:2; even more preferably from 1.5:1 to 1:1.5, still more preferably from 1.3:1 to 1:1.3, and most preferably from 1.2:1 to 1:1.2.
9. The method or apertured nonwoven web of any of the preceding examples wherein the fibrous nonwoven web comprises bicomponent fibers.
10. The method or apertured nonwoven web of example 9 wherein the bicomponent fibers are of a sheath-core bicomponent configuration having a sheath component and a core component.
11. The method or apertured nonwoven web of example 10 wherein the core component comprises a polymer selected from the group consisting of PET, PP and PE and combinations thereof, and preferably, PET.
12. The method or apertured nonwoven web of either of examples 10 or 11 wherein the sheath component comprises PE, preferably predominantly PE.
13. The method or apertured nonwoven web of any of the preceding examples, wherein constituent fibers of the fibrous nonwoven web comprise fibers that are, in weight proportion, predominantly, substantially, or entirely hydrophobic, or rendered hydrophobic via fiber surface finish.
14. The method or apertured nonwoven web of any of the preceding examples wherein the fibrous nonwoven web (10) includes a first layer component having a first fiber constitution and a second layer component having a second fiber constitution differing from the first fiber constitution.
15. The method or apertured web of example 14 wherein the first layer comprises predominantly hydrophobic fibers and the second layer comprises predominantly hydrophilic fibers.
16. The method or apertured nonwoven web of either of examples 14 or 15 wherein the second fiber constitution comprises a combination of bicomponent fibers and hollow monocomponent fibers.
17. The method or apertured nonwoven web of example 16 wherein the hollow monocomponent fibers comprise PET.
18. The method or apertured nonwoven web of any of examples 14-17 wherein the second fiber constitution comprises cellulosic fibers.
19. The method or apertured nonwoven web of example 18 wherein the cellulosic fibers comprise regenerated cellulose.
20. An apertured nonwoven web manufactured by the method of any of examples 1-3 or 9-19.
21. A feminine hygiene pad comprising a liquid-permeable topsheet, a liquid-impermeable backsheet, and an absorbent structure disposed between the topsheet and the backsheet, wherein the topsheet comprises the apertured nonwoven web of any of the preceding examples.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A single step method for imparting a pattern of apertures in a fibrous nonwoven web constituted predominantly of staple fibers, comprising the steps of:
providing a pinned roller having a first axis and bearing a pattern of aperturing pins extending radially outwardly therefrom, wherein the aperturing pins have radially outermost top surfaces which are substantially smooth and lie along a cylindrical shape profile about the first axis, the top surfaces having perimeter edges thereabout that define surface areas of the top surfaces;
providing an opposing roller having a second axis;
arranging the opposing roller and the pinned roller so as to form a nip therebetween, wherein the first and second axes are substantially parallel, and wherein the nip has effectively zero clearance between the top surfaces of the aperturing pins and one or more outermost surfaces of the opposing roller,
wherein the opposing roller comprises one or more radially outermost surfaces that is/are cylindrical about the second axis, and disposed on the opposing roller so as to opposingly contact the top surfaces of the aperturing pins in the nip;
providing heating energy to one or both of the pinned roller and the opposing roller;
rotating the pinned and opposing rollers, and conveying the fibrous nonwoven web through the nip,
whereby material(s) of which fibers of the nonwoven web are constituted is/are at least partially melted and expressed from nip regions between the top surfaces of the pins and the opposing roller when the pins meet the opposing roller in the nip, outwardly towards the perimeter edges of the pin top surfaces, and
whereby expressed fiber material accumulates to form densified zones about the perimeter edges of the top surfaces of the pins, that remain with the nonwoven web as it exits the nip;
whereby a pattern of open apertures is imparted to the web, the pattern of apertures corresponding to the pattern of aperturing pins, thereby providing an apertured nonwoven web.

2. The method of claim 1 wherein the perimeter edges of the outermost top surfaces of the aperturing pins define shapes having an average aspect ratio of from 2.5:1 to 1:2.5; more preferably 2:1 to 1:2; even more preferably from 1.5:1 to 1:1.5, still more preferably from 1.3:1 to 1:1.3, and most preferably from 1.2:1 to 1:1.2.

3. The method of claim 1 further comprising the step of air-through bonding the fibrous nonwoven web or apertured nonwoven web, either prior to or subsequent to the step of conveying the fibrous nonwoven web through the nip.

4. The method of claim 1 wherein the fibrous nonwoven web comprises bicomponent fibers.

5. The method of claim 4 wherein the bicomponent fibers are of a sheath-core bicomponent configuration having a sheath component and a core component.

6. The method of claim 1, wherein constituent fibers of the fibrous nonwoven web comprise fibers that are, in weight proportion, predominantly, substantially, or entirely hydrophobic, or rendered hydrophobic via fiber surface finish.

7. The method of claim 1 wherein the fibrous nonwoven web includes a first layer component having a first fiber constitution and a second layer component having a second fiber constitution differing from the first fiber constitution.

8. The method of claim 7 wherein the first layer comprises predominantly hydrophobic fibers and the second layer comprises predominantly hydrophilic fibers.

9. An apertured nonwoven web comprising staple fibers, having a pattern of apertures therethrough, wherein each of a plurality of the apertures is surrounded by a densified agglomeration of material(s) of which fibers of the nonwoven web are composed, the materials having been plastically deformed and/or fused via z-application of localized z-direction direction compression and optionally, application of heat.

10. The apertured nonwoven web of claim 9, wherein the densified agglomeration of materials is relatively larger and/or more dense in a first region proximate a first portion of a perimeter said each of said plurality of apertures, and relatively smaller and/or less dense in a second region proximate a second portion of the perimeter of said each of said plurality of apertures, wherein the first portion of the perimeter is oppositely disposed of the second portion of the perimeter.

11. The apertured nonwoven web of claim 10 wherein the first portion of the perimeter is disposed on an upstream side of said each of said plurality of apertures, and the second portion of the perimeter is disposed on a downstream side of said each of said plurality of apertures, wherein “upstream” and “downstream” are relative a machine direction along which the apertured nonwoven web was passed through a nip between a pair of aperturing rollers.

12. The apertured nonwoven web of claim 9, comprising a plurality of randomly distributed fiber-to-fiber bonds therewithin, the fiber-to-fiber bonds not exhibiting effects of z-direction compression in the formation thereof.

13. The apertured nonwoven web of claim 9, wherein the apertures in the pattern have an average aspect ratio of from 2.5:1 to 1:2.5; more preferably 2:1 to 1:2; even more preferably from 1.5:1 to 1:1.5, still more preferably from 1.3:1 to 1:1.3, and most preferably from 1.2:1 to 1:1.2.

14. The apertured nonwoven web of claim 9 wherein the fibrous nonwoven web comprises bicomponent fibers.

15. The apertured nonwoven web of claim 14 wherein the bicomponent fibers are of a sheath-core bicomponent configuration having a sheath component and a core component.

16. The apertured nonwoven web of claim 9, wherein constituent fibers of the fibrous nonwoven web comprise fibers that are, in weight proportion, predominantly, substantially, or entirely hydrophobic, or rendered hydrophobic via fiber surface finish.

17. The apertured nonwoven web of claim 9 wherein the fibrous nonwoven web includes a first layer component having a first fiber constitution and a second layer component having a second fiber constitution differing from the first fiber constitution.

18. The apertured nonwoven web of claim 17 wherein the first layer comprises predominantly hydrophobic fibers and the second layer comprises predominantly hydrophilic fibers.

19. An apertured nonwoven web manufactured by the method of claim 1.

20. A feminine hygiene pad comprising a liquid-permeable topsheet, a liquid-impermeable backsheet, and an absorbent structure disposed between the topsheet and the backsheet, wherein the topsheet comprises the apertured nonwoven web of claim 9.
